(19) United States
(12) Patent Application Publication
(10) Pub. No.: US 2003/0187592 A1
(43) Pub. Date: Oct. 2, 2003

(54) Association rule mining and visualization for disease related gene

(75) Inventors: Yoshihiro Ohta, Tokyo (JP); Tetsuo Nishikawa, Tokyo (JP); Shigeo Ihara, Tokorozawa (JP)

Correspondence Address: Stanley P. Fisher, Reed Smith LLP, Suite 1400, 3110 Fairview Park Drive, Falls Church, VA, 22042-4503, US

(73) Assignee: Hitachi, Ltd.

(21) Appl. No.: 10/309,141

(22) Filed: Dec. 4, 2002

(30) Foreign Application Priority Data
Mar. 26, 2002 (JP) 2002-085962

(51) Int. Cl. G06F 19/00; G01N 33/48; G01N 33/50

(52) U.S. Cl. 702/20

(57) ABSTRACT

Features relating to gene expression levels are extracted and visualized in an effective manner. Data about the gene expression levels is utilized for medical diagnosis. Information about whether or not the expression levels of each of positive samples are within a predetermined range is displayed comparatively with information about whether or not the expression levels of each of negative samples are within a predetermined range.

### BACKGROUND OF THE INVENTION

1. Technical Field

The present invention relates to a display system for extracting features of genes in a group of samples with a certain property and those in another group of samples without that property, deriving the difference between the two groups and displaying it on a screen. The invention also relates to a medical diagnostic support system for determining to which group a new sample is more likely to belong.

2. Background Art

The DNA microarray technique makes it possible to monitor the expression levels of a large number of genes at once. The expression level of a particular gene is believed to be closely related to biological phenomena in the individual that has the gene. It is expected that by analyzing gene expression levels, light can be shed on the behavior of genes responsible for a variety of biological phenomena. Expectations are particularly high for diagnostics, treatment and creation of new drugs based on the identification of genes responsible for diseases that are believed to have a genetic cause.

The genes to be analyzed number in the thousands, of which it is believed only a few are related to genetic diseases. If all possible combinations of the few genes selected from the thousands of genes are to be examined, that would add up to tremendous numbers and the work required would not be finished in a realistic period of time. Thus, an algorithm is required that enables useful features to be obtained efficiently.

There are roughly two types of expression level analysis methods. One is a machine learning technique using support vector machines (Terrence S. Furey, Nello Cristianini, Nigel Duffy, David W. Bednarski, Michel Schummer, David Haussler, “Support Vector Machine Classification and Validation of Cancer Tissue Samples Using Microarray Expression Data”). In this method, a new sample is evaluated to determine which classification it belongs to based on the process of learning based on previously classified cell samples. This method classifies cells, for example, into either a group that has a disease or a group that does not, thus providing a diagnostic system. While this method can determine whether a particular cell has a certain disease or not, it cannot clarify which gene or genes are responsible for the disease.

The other expression level analysis method that is attracting attention is data mining. Data mining is used to extract correlations from a large database of products, for example, purchased by customers. Correlations are derived by determining significant rules using rule-evaluation measures called support and confidence. Algorithms for efficiently extracting rules satisfying support and confidence are given by R. Agrawal, T. Imilienski, and A. Swami, “Mining Association Rules Between Sets of Items in Large Databases” and Sergey Brin, Rajeev Motwani, Jeffrey D. Ullman, and Shalom Tsur, “Dynamic Itemset Counting and Implication Rules for Market Basket Data.”

However, the measurement of expression levels by the DNA microarray method is so costly that expression level data for a great number of samples cannot be obtained. On the other hand, when the number of samples is small, the data mining method cannot easily determine the rules that satisfy support and confidence.

It is widely known that in many cases the information contained in genes plays a big role in influencing a person's susceptibility to a certain disease or how well a drug will work, for example. By taking full advantage of the information about gene expression levels obtained by the microarray method, diseases can be prevented or more effective treatment methods can be selected, for example. Thus, many researchers are trying to find more effective methods of extracting gene features. Particularly, extracting differences between a group with a certain property and another group without that property is more effective than by examining only the genes of the group with the property. Accordingly, a method is required that allows extraction of features that are exhibited strongly in one group but which are scarcely exhibited in another group. Those features are known to generally be expressed by combinations of a plurality of genes. When more than 10,000 genes are involved, staggering numbers of calculations and amounts of memory would be required for extracting the features. Furthermore, the extracted features would be so numerous that they could not be easily and effectively visualized.

It is therefore an object of the invention to provide a system for displaying extracted features that can effectively reduce the required amount of calculation and memory. It is another object of the invention to provide a medical diagnostic support system for determining to which group a tested sample is more likely to belong.

### SUMMARY OF THE INVENTION

In the present specification, a group from which features are to be extracted is referred to as a Positive Group, and a reference group is referred to as a Negative Group. Examples of the criteria on which basis the groups are divided include:

(1) Whether or not the patient is afflicted with a certain disease;

(2) Whether or not the patient survived three years or more after surgical operation;

(3) Whether or not a particular medicine proved effective; and

(4) Whether or not a tumor metastasized after radiation therapy.

In the case of example (1), samples afflicted with a certain disease are classified as Positive Group and those that are not afflicted with the disease are classified as Negative Group. In the case of example (3), samples which showed improvements after administration of a medicine are classified as Positive Group and samples which showed no improvements are classified as Negative Group.

The concept of the present invention can be also applied to analysis using protein chip technology, whose operational principle is the same as that of the DNA microarray method. Protein chips are designed to examine the working of proteins, which are produced according to DNA information. For example, the antibody of a protein is attached to the chip so that information about a particular protein can be obtained by using laser or the like, taking advantage of the protein's tendency to bind to the particular antibody.

In the following, various embodiments of the invention are listed.

(1) A display system for displaying information about expression levels of genes in samples, wherein information about expression levels of each of a plurality of samples belonging to a first group is displayed comparatively with information about expression levels of each of a plurality of samples belonging to a second group having a different property from that of the first group.

(2) The display system according to embodiment (1), wherein items of information about expression levels of a plurality of genes are displayed in a comparative manner.

(3) The display system according to embodiment (1), wherein the information about the expression levels indicates whether or not the expression levels are within a predetermined range.

(4) The display system according to embodiment (3), wherein the first group has a specific property (Positive Group) and the second group does not have the predetermined property (Negative Group).

(5) The display system according to embodiment (1), wherein items of information about the expression levels of a plurality of samples belonging to the first group are displayed adjacent to one another, and items of information about the expression levels of a plurality of samples belonging to the second group are displayed adjacent to one another.

(6) A display system for displaying information about expression levels of genes in samples, wherein two histograms are displayed, the first histogram being prepared on the basis of the expression levels of each of the samples belonging to a first group, and the second histogram being prepared on the basis of the expression levels of each of the samples belonging to a second group that has a different property from that of the first group, each histogram having one axis showing expression levels and the other axis showing the number of samples.

(7) The display system according to embodiment (6), wherein the first and second histograms are displayed in a superposed manner on a single graph sharing the same axes.

(8) The display system according to embodiment (7), wherein the first and second histograms are displayed in different manners, for example with different colors or shades, so that the individual histograms can be clearly identified even when they are superposed.

(9) The display system according to embodiment (6), wherein the axis showing the expression levels is divided into a plurality of expression level sections.

(10) The display system according to embodiment (6), wherein the first group has a certain property (Positive Group) and the second group does not have that property (Negative Group).

(11) A medical diagnostic support system comprising:

a storage unit for storing a set of combinations of expression level ranges of a plurality of genes characterizing the presence of a certain property, and a set of combinations of expression level ranges of a plurality of genes characterizing the absence of the property;

a computing unit for calculating the probability of a tested sample having the property of interest by comparing the expression level ranges of a plurality of genes in the sample with the combinations of expression level ranges of the plurality of genes stored in the storage unit; and

a display unit for displaying the result of computation in the computing unit.

(12) The medical diagnostic support system according to embodiment (11), wherein the property relates to the fact that a certain treatment method is effective.

(13) The medical diagnostic support system according to embodiment (11), wherein the property relates to the fact that the sample is afflicted with a certain disease.

(14) The medical diagnostic support system according to embodiment (11), wherein the property relates to the fact that the sample is prone to a certain disease.

(15) The medical diagnostic support system according to embodiment (11), wherein the result of computation in the computing unit is displayed on the display unit in numerical terms.

(16) The medical diagnostic support system according to embodiment (11), wherein the result of computation in the computing unit is displayed on the display unit in percentage terms on a graph.

(17) The medical diagnostic support system according to embodiment (11), wherein the storage unit for storing a set of combinations of expression level ranges of a plurality of genes characterizing the presence of a certain property, and a set of combinations of expression level ranges of a plurality of genes characterizing the absence of the property is provided for each of a plurality of different properties.

(18) The medical diagnostic support system according to embodiment (17), wherein the computing unit calculates the probability of the tested sample having a first property by comparing the expression level ranges of a plurality of genes in the tested sample with combinations of expression level ranges of a plurality of genes stored in a first storage unit, and calculates the probability of the tested sample having a second property by comparing the expression level ranges of a plurality of genes in the tested sample with combinations of expression level ranges of a plurality of genes stored in a second storage unit.

(19) The medical diagnostic support system according to embodiment (18), wherein the display unit displays the probability of the tested sample having the first property and the probability of it having the second property.

(20) The medical diagnostic support system according to embodiment (18), wherein the display unit displays the probability of the tested sample having the first property and the probability of it having the second property in percentage terms on a graph.

### BRIEF DESCRIPTION OF THE DRAWINGS

FIG. 1 shows a method of converting data.

FIG. 2 shows the effect of data reduction.

FIG. 3 shows a search tree.

FIGS. 4A and 4B show algorithms for judging rules.

FIG. 5 shows a diagnostic system.

FIG. 6 shows an example of classification by ontology.

FIG. 7 shows a viewer window displaying a list of rules that were extracted.

FIG. 8 shows an example of a visualization of a rule.

FIG. 9 shows an enlarged view of sample distribution.

FIG. 10 shows an example of a viewer window for gene-related articles.

FIGS. 11 shows an example of a viewer window for the nucleotide sequence of a gene.

FIG. 12 shows an example of a visualization of genes arranged in an order of importance.

FIG. 13 shows an example of a visualization of a network formed by degrees of gene connection.

FIG. 14 shows an example of visualization of a network formed by degrees of gene connection in association with a network formed by correlations of genes appearing in literature.

FIG. 15 shows an example of the system according to the invention.

### DETAILED DESCRIPTION OF PREFERRED EMBODIMENTS

The invention will be hereafter described by way of embodiments with reference made to the attached drawings.

1. Data Conversion

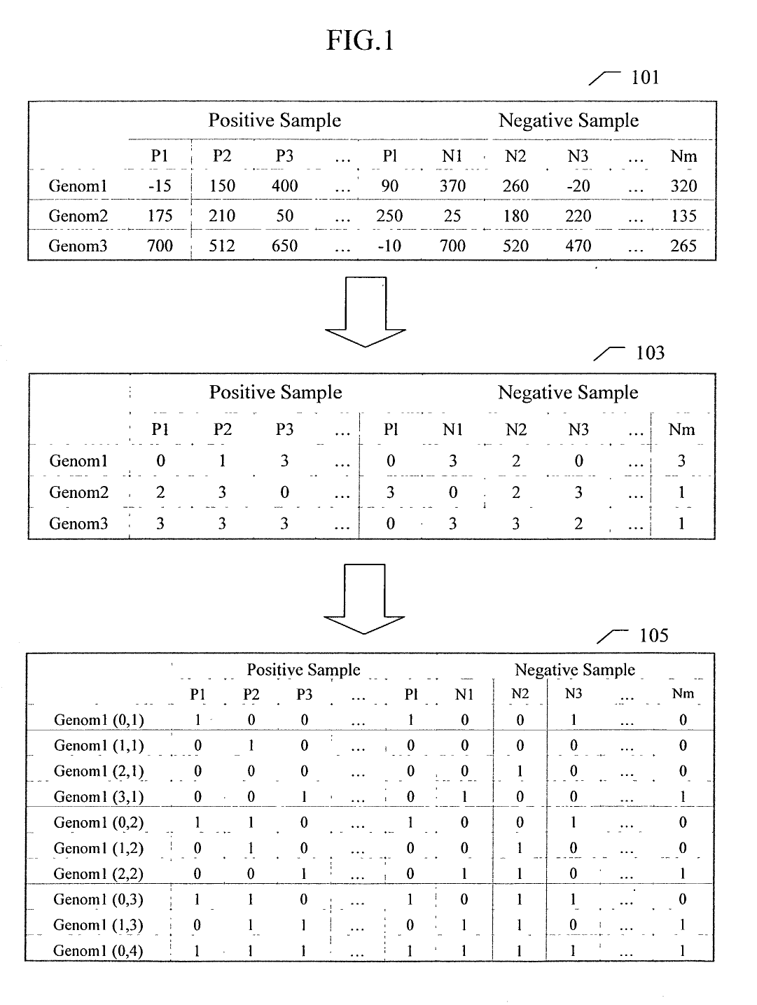

Data is given as real values of the expression levels of individual genes in a group having a certain property and a group having no such property. A more detailed description of data will be given in section 1. 1. In section 1.2, a method of converting the real-value data into discrete-value data that is suitable for retrieval and extraction of features will be outlined. Finally, a method of converting the discrete-value data into binary data that is more suitable for retrieval and extraction of features will be described in section 1.3. Specific examples are shown in FIG. 1.

1.1 Data Form

Data is given as real values of expression levels of individual genes in the samples in a group having a certain property and the samples in another group having no such property (Table 101). In Table 101, P1, . . . P1 indicate samples with a certain property from a number 1 of people (Positive). N1, . . . , Nm indicate samples without that property from a number m of people (Negative). The genes are identified by numbers, such as Genome1, Genome2, Genome3 and so on for simplicity's sake, although actually they have their own names.

The expression levels of approximately 10,000 genes are given for each sample in each group. The values of the expression levels may range from negative values to the thousands, for example, depending on the analysis method used. It is not yet exactly known, however, what meaning those values have as absolute amounts. Thus, these values must be evaluated in relative terms so that they can be given a meaning. In the case of the data example shown, it is assumed that the only reference for absolute evaluation is the following:

Regard Values of 100 or Less as 0

This is due to the known fact that values of around 100 are sometimes produced by instrumental problems even when there is no expression at all of a particular gene. The following fact is also known:

Values of 100 or More do not Exceed Actual Expression Levels

Thus, when there are two or more items of data for the same sample or gene, the larger item is adopted.

This data, continuous-value data, is not suitable for data retrieval or feature extraction. The reason is that the continuous-value data requires huge amounts of calculation and memory during data retrieval and feature extraction. Thus, in the subsequent sections, a method of converting the continuous-value data into a format suitable for data retrieval and feature extraction will be described.

1.2 Conversion Into Discrete Values

As mentioned in section 1.1, the data is given as continuous-value data, which is not suitable for data retrieval or feature extraction. So the data has to be converted into discrete-value data by some method. However, conversion of continuous-value data into discrete-value data creates data degradation. Some conversion methods can cause considerable deterioration in data, making it difficult to extract features accurately. For example, the following method can cause significant deterioration in data.

(Example) A method that sets a threshold and converts values smaller than the threshold into zero and values larger than the threshold into one.

This conversion method has the following two problems:

(1) How to determine the threshold value.

(2) Inability to extract features concentrated in a certain section or sections.

As for problem 1, it is very difficult to set an appropriate threshold value. For instance, if the threshold is too large, most of the expression levels would be converted into zero and features that should have been extracted may not be extracted. If the threshold is too small, most of the expression levels would be converted into one, and more features than necessary could be extracted meaninglessly. In another case, the mean of all the values could be adopted as the threshold value. In this case, about half of the total will be one and the remaining half zero, making it such that the data has no features.

Even if problem 1 is solved and an appropriate method of determining the threshold is established, problem 2 remains. In the methods using the threshold, evaluation is based only on whether a particular expression level is larger or smaller than a certain value. In many cases, however, the data given contains a small number of samples having extremely large or small expression levels, with the rest concentrated in a certain section or sections. Such important features cannot be extracted by methods using a single threshold value.

In the following, a method of converting the data into discrete values by setting a number n of boundaries will be described as a means of solving problems 1 and 2.

As mentioned in section 1.1, in the illustrated example, expression levels of 100 or less can be regarded as zero. Thus, the interval between 100 and the maximum value of the expression levels is equally divided into n sections, and the sections are given boundary values b₁ . . . bₙ. Based on the boundary values, the continuous values in each section are allotted discrete values from 0 to n. This is done by means of a function called Border.

$$Border ( x ) = \{ \begin{matrix}0 & ( x \leq {b}_{1} ) \\ 1 & ( {b}_{1} < x \leq {b}_{2} ) \\ 2 & ( {b}_{2} < x \leq {b}_{3} ) \\ ⋯ & \\ n & ( {b}_{n} < x )\end{matrix}$$

By using this function, the continuous-value data given (Table 101) is converted into discrete-value data (Table 103).

The method of converting the continuous-value data into discrete-value data by Border (x) solves both problems encountered when converting with one threshold value. Namely, with regard to the first question of how to select the threshold value, since the present method uses more than one boundary value, the values selected as the boundary values influence the data far less than the one threshold does. Although the influence can be further reduced as desired by increasing the number n of the boundaries, the number must be appropriately set for individual environments by taking into consideration the trade-off between an increase in n and the resultant increase in the cost of calculations and memory. However, experiments show that increasing the value of n does not necessarily result in too great an increase in costs, as will be described in section 3.1, thus proving the effectiveness of this conversion method. As for problem 2, this problem is obviously solved, since in this method, data concentrated in certain sections will manifest themselves also in certain sections, such as sections 3 to 5 for example. in the post-conversion data.

1.3 Conversion Into Binary Values

The discrete-value data obtained in section 1.2 is more suitable for data retrieval and feature extraction than the continuous-value data. But in order to use a concept called support for performing fast feature extraction, as will be described in section 2.3, the data must be given in binary values. Thus, in this section, a method of converting the discrete-value data (Table 103) obtained in 1.2 into binary data (Table 105) will be described.

The individual values of the discrete-value data (Table 103) obtained in 1.2 are approximations of the continuous values in the original data. An example of the feature to be extracted from this data might show that a certain gene in a group with a certain property is expressed at high levels within certain ranges in a concentrated manner.

(Example) For a group with a certain property, 90% or more of the samples have values from 3 to 5 for gene 3.

This feature can be extracted by the following binary data for example:

$$f ( x ) = \{ \begin{matrix}1 & ( 3 \leq x \leq 5 ) \\ 0 & ( otherwise )\end{matrix}$$

The binarization by this function differs greatly from the binarization by means of a threshold described in 1.2. If appropriate binarization can be performed in this way, features can be extracted accurately. However, in reality it cannot be known in advance in what range a particular gene is expected at high levels. Thus, a method is considered that converts expression levels in each of the different sections into binary data. and the following equation is introduced for this purpose:

(i,j)={x|i≦x≦i+j−1}

By varying i and j from 1 to n, all sections can be covered. It will be seen that the total number of sections is, when there are n+1 sections with width 1 and n sections with width 2, given by

$$\frac{( n + 1 ) ( n + 2 )}{2}$$

For each of the sections thus created, a binary value is assigned on the basis of whether the discrete-value data of Table 103 is in the section. Namely, the following conversion is performed:

$${Binary}_{( i , j )} ( x ) = \{ \begin{matrix}1 & ( i \leq x \leq i + j - 1 ) \\ 0 & ( otherwise )\end{matrix}$$

It can be seen that the above f(x) is similar to Binary_{(3,3)}(x).

Table 105 shows binary data obtained by converting the discrete-value data relating to Genome 1 of Table 103 using the above conversion. It should be noted here that one line of data for each gene is converted into [(n+1)(n+2)/2] lines of data. This is because each item of discrete-value data is binarized with varying sections. As a result, it appears as if the data is multiplied by [(n+1)(n+2)/2]. In substance, however, the amount of data is not changed. In addition, as will be shown in 3.1, only part of the data of Table 105 is used for the actual extraction of features, so the problem of the data amount increasing by the order of magnitude of n squared does not occur in reality.

Each line of data (Table 105) obtained by the conversion described in this section consists of binary data indicating whether an expression level of a certain gene belongs to a certain section. Namely, when some kind of feature is extracted for this data, this means that a feature relating to a gene and an expression level section have been extracted.

2. Definition of the Value Reference of Features

Before subjecting the data converted in section 1 to feature extraction, the term features must be accurately defined. Thus, desirable features to be extracted, chosen after considering a given objective, will be described in section 2.1. Section 2.2 introduces the term “rule,” and further defines the term feature. Finally in section 2.3. the value reference for the rule will be defined.

2.1 Required Features

The features to be extracted by the method of the invention are used in determining whether a new sample is more likely to belong to a group with a certain property or another group without that property. Namely, the extracted features should indicate the difference between the group with the property and the group without that property. Thus, the required feature can be described as follows:

Required Feature

The feature shows that “if a gene in a sample has that feature, it is likely that the sample has (or does not have) a certain property.”

For example, with regard to the data in the line of Genome1(0,2) in Table 105, there are more is in the samples with a certain property and there are more 0s in the samples without that property. Namely, it will be seen that it is likely that a sample having a gene expression level in this section has this property. In other words, this gene section can be a required feature.

However, it is known that in general, such a genetic feature is caused by more than one gene. Thus, the same procedure is repeated for a combination of lines of the data of Table 105. Accordingly, in the subsequent sections, a combination of the expression level sections of genes is defined as a rule, and conditions for a rule to become a required feature will be described.

2.2 Rule

It is known that whether a certain property is present or not depends generally on a plurality of genes. Thus, it is necessary to perform extraction of features on a combination of different lines from the data in Table 105. In this section, this combination is accurately defined.

Initially, each line of Table 105 is regarded as a function for assigning a binary value to each sample. A function r representing each line can be expressed by:

r : Positive∪Negative→{0,1}

wherein Positive designates a set of samples having a certain property, and Negative designates a set of samples not having the property. For example, when the function corresponding to line 1 is r₁, we have:

r₁(P₁)=1

r₁(P₂)=0

r₁(N₁)=0

r₁(N₂)=0

Likewise, when a function corresponding to line 2 is r₂, we have:

r₂(P₁)=0

r₂(P₂)=1

r₂(N₁)=0

r₂(N₂)=0

This can be accurately defined as follows:

$${r}_{i} ( P ) = \{ \begin{matrix}1 & ( sample P has the i th property ) \\ 0 & ( sample P does not have the i th property )\end{matrix}$$

A rule is defined as a set of functions representing the individual lines thus defined above. For example, {r₁, r₂} and {r₁, r₂, r₅, r₆, r₉} are rules. A set consisting of a single element (such as {r₁} and {r₂}) is also a rule.

The rule is a combination of functions representing the individual lines in table 105. Namely, the rule is a combination of sections of individual genes. It should be noted that lines representing different sections of the same gene should not be present in a single rule simultaneously. This is because successive sections are all covered during the conversion into binary values, and the above mixture results in data redundancy. For example, when function Genome1 (0, 1) corresponding to line 1 and function Genome1 (1, 1) corresponding to line 2 of Table 105 are combined to form a rule, it will be seen that this is the same as function Genome1 (0, 2) corresponding to line 5. To avoid such situations, it is assumed that each rule can contain one line for each gene. This assumption will be omitted when making rules by an algorithm.

In the next section, a value standard for the thus defined rules will be defined and a method of determining which rule can become a required feature will be described.

2.3 Value Standard for the Rules

The feature that is required should indicate that “when genes of a sample have that feature, it is likely that the sample has (or does not have) a certain property. Namely, it will be seen that this can be translated into the situation in which one group has a high probability of occurrence of 1 while the other group has a low probability of occurrence of 1. To indicate “the probability of occurrence of 1,” the concept of support is introduced.

(Definition) Support

spt_{Positive}(R)={s ε Positive|r(s)=1 for all r ε R}

spt_{Negative}(R)={s ε Negative|r(s)=1 for all r εR}

sptp(R) and spt_{N}(R) are each a set of those samples in the Positive (Negative) set of samples, for which all of the lines belonging to R are 1. For example, when a set of function Genome1 (1, 3) corresponding to line 9 and function Genome1 (0, 4) corresponding to line 10 of Table 105 is assumed to be a rule, we have:

spt_{Positive}(R)={P2,P3}

spt_{Negative}(R)={L1,L2,Lm}

As a result, it will be seen that the greater the number of the elements in a set defined by support, the greater the probability that the lines in the group are 1. Next, as an indicator of the difference between the two groups. differential confidence will be defined. #A designates the number of elements in set A.

(Definition) Differential Confidence

$$conf ( R ) = \frac{\# {spt}_{Positive} ( R )}{\# {spt}_{Positive} ( R ) + \# {spt}_{Negative} ( R )}$$

Thus, differential confidence indicates, of all the samples that are 1, the proportion of those samples belonging to Positive Group. It will be seen that the greater this value is, the greater the difference between the probability of occurrence of 1 in Positive Group and that of occurrence of 1 in Negative Group. Namely, it will be seen that a rule with a large differential confidence can be the required feature. The thus defined differential confidence represents the level of confidence for the differentiation for the two sets. which is different from the conventional concept of confidence.

From the above discussion, it will be seen that searching for a rule with high differential confidence is important for the extraction of a strong feature. But in reality, this criterion is not sufficient for the extraction of a good feature.

For example:

(Example) Assume that the following two are valid rules giving a high probability that a sample belong to Positive Group and not Negative Group.

(1) When genes 1 and 2 of a subject are expressed, the probability is high that the subject belongs to Positive Group and does not belong to Negative Group.

(2) When genes 1, 2 and 3 of a subject are expressed, the probability is high that the subject belongs to Positive Group and does not belong to Negative Group.

In this case, it will be seen that feature (1) suffices. Namely, it will be seen that if a partial rule of a certain rule realizes the same level of differential confidence as that of the original rule, only the partial rule should be extracted as a feature.

It will be seen from the definition of support that as the number of elements in a rule increases, #spt_{Positive}(R) or #spt_{Negative}(R) will never increase but will gradually decrease. In order to increase differential confidence, #sptₚₒₛᵢₜᵢᵥₑ(R) must be increased and #spt_{Negative}(R) must be decreased. Thus, in order to realize a high level of differential confidence with as small a rule as possible, it is essential to decrease #spt_{Negative}(R) in an efficient manner. By taking this into consideration, a rule that does not contain unwanted rules (the most minimum rule that will realize the potential of that rule), that is a minimum gene rule, is defined below. This definition is based on the understanding that a smaller rule has more utility value when there are alternative rules with the same value.

(Definition) Minimum Gene Rule

Rule R is said to be a minimum gene rule when it is such that, with respect to every partial rule R′(R′⊂R, R′≠R),

#spt_{Negative}(R′)>#spt_{Negative}(R)

The minimum gene rule is a very effective concept for finding a small rule for realizing a high differential confidence. But to determine whether or not a rule is a minimum gene rule, each and every partial rule must be examined. Specifically, calculations on the order of magnitude of the square of the number of elements in the rule would have to be performed. An accelerated rate of calculation can be ensured by using the following theorem:

(Theorem 1) With regard to rule R, the following two are equivalent:

(i) With respect to each and every partial rule R′(R′⊂R, R′≠R) of rule R.

#spt_{Negative}(R′)>#spt_{Negative}(R)

(ii) With respect to such sets of partial rules R′(R′⊂R, R′≠R) of rule R that #R′=#R−1,

#spt_{Negative}(R′)>#spt_{Negative}(R)

According to this theorem, it will be seen that whether a particular rule is a minimum gene rule or not can be determined by examining not all but only those partial rules with the number of elements less by one. Namely, the minimum gene rule can be judged based on an amount of calculations of the order of magnitude of the number of elements in the rule raised to the first power.

The minimum gene rule concept is useful not only for the extraction of features with higher values but also for reducing the number of calculations required for feature extraction. This is ensured by the following theorem.

(Theorem 2) When rule R′ is not a minimum gene rule, rule R′(R′⊂R) that includes it as a partial rule is not a minimum gene rule either.

According to this theorem, when rules are created by an algorithm, it is not necessary to increase the number of elements in a rule that is not the minimum gene rule and such a rule can be disposed of at that stage. Thus, wasteful calculations can be avoided and the number of calculations can be greatly reduced.

It will be seen from the descriptions so far that a minimum gene rule with a high differential confidence can be a required feature. However, there are two points about the differential confidence that should be carefully considered if a minimum gene rule is to become a required feature.

One is the fact that, while differential confidence represents, of all of the samples that are 1, the proportion that belongs to Positive Group, this indicator does not show the entire number of samples that are 1. Accordingly, in the case where there is only one sample belonging to Positive Group and no samples belong to Negative Group, differential confidence exhibits a maximum value even though there is no real significance in this differential confidence. To avoid such a scenario, a lower limit Border_{Positive} is assigned to #spt_{Positive}(R). Similarly, an upper limit Border_{Negative} is assigned to #spt_{Negative}(R). Thus, a lower limit is virtually given to differential confidence.

The second point is that, as mentioned in the discussion of the minimum gene rule, it is desirable that the rule to be extracted should be as small as possible. On the other hand, if a rule that constitutes a required feature is defined along the line of discussion made so far, there is the possibility that the thus defined rule to which a further rule has been added may become a rule constituting the required feature. To avoid this, a new condition is introduced which says a partial rule shall not be a rule constituting a required feature.

Based on the above discussion, the rule constituting a required feature is defined as a disease causation rule in the below-indicated manner. This is in order to weed out rules with lower values before arranging all of the rules in a certain order, as the number of the rules that are extracted is huge.

(Definition) Disease Causation Rule

Rule R is said to be a disease causation rule when it satisfies the following four conditions with respect to Border_{Positive} and Border_{Negative} given.

(1) R is a minimum gene rule.

(2) With respect to R′(R′⊂R, R′≠R), #spt_{Negative}(R′)≧Border_{Negative}.

(3) #spt_{Positive}(R)≧Border_{Positive}

(4) #spt_{Negative}(R)<Border_{Negative}

In a range satisfying the condition #spt_{Positive}(R)≧Border_{Positive}, the relationship between differential confidence of a disease causation rule and that of a non-disease causation rule must be carefully considered. With regard to this relationship, the following theorem is known:

(Theorem 3) A necessary and sufficient condition for a minimum value of differential confidence of a disease causation rule to be larger than a maximum value of differential confidence of another rule that satisfies #spt_{Positive}(R)≧Border_{Positive} is given by the following inequality:

Border_{Positive}Border_{Negative}−(Border_{Negative}−1)l>0

where 1 is the number of samples in Positive Group.

This condition can be satisfied by setting Border_{Positive} at a large value and Border_{Negative} at a small value. In fact, in order to increase the differential confidence of a rule extracted as a disease causation rule, a large value and a small value must be assigned to Border_{Positive} and Border_{Negative}, respectively. By so doing, the condition of Theorem 3 is satisfied.

There are also conditions for a sub-rule to the thus defined disease causation rule, as in the case of the minimum gene rule. By using these conditions, the amount of calculation can be reduced. This is ensured by the following theorem:

(Theorem 4) Rule R is not a disease causation rule if rule R′(R′⊂R, R′≠R) does not satisfy any of the following conditions:

(1) Rule R′ is a minimum gene rule.

(2) #spt_{Positive}(R′)≧Border_{Positive.}

(3) #spt_{Negative}(R′)≧Border_{Negative.}

It will be seen that, from this theorem, a rule that does not satisfy any of the conditions (1) to (3) need not be combined with any other rules and may instead be deleted at that point. Thus, unnecessary calculations can be avoided and the amount of calculation can be greatly reduced.

The feature represented by the thus defined disease causation rule is valuable in that it indicates that if genes in a sample have that feature, the probability is high that the sample has (or does not have) a certain property. Also, the feature is made up of a minimum combination of elements for realizing that value.

3. Algorithm for Extracting Rules

A description of an algorithm for searching for all the rules that can be the disease causation rule as defined in section 2.3 will follow. In section 3.1, a description will be given about how, from the binary data (Table 105) obtained by converting continuous-value data given, only those lines that can constitute a disease causation rules are selected in order to reduce the amount of data. In section 3.2, an algorithm will be described for creating a disease causation rule by combining items of the reduced data.

3.1 Data Reduction

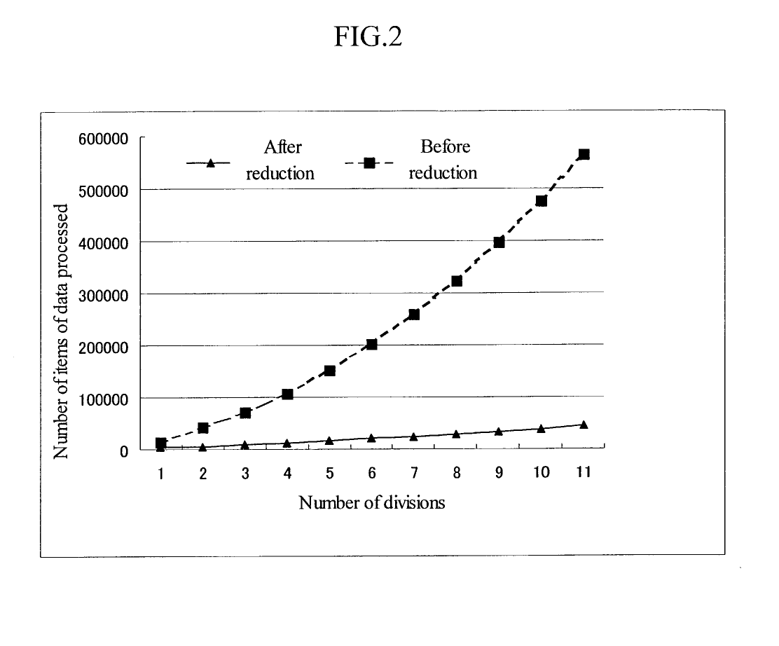

Each line of the binary data obtained by converting the continuous-value data given may be regarded as a rule with a single element. Thus, of those rules, ones that do not satisfy the conditions required of a partial rule to the disease causation rule can be deleted in advance, thereby greatly reducing the amount of data. FIG. 2 shows the difference in the amount of data between a case where sections of unnecessary gene expression levels have been eliminated, and a case where they have not been eliminated. In the drawing, the lateral axis shows the number of divisions, and the vertical axis shows the number of items of data to be processed. The data used here is based on the expression levels of 7220 genes from two groups of 16 patients, one group having features related to cancerous diseases and the other group having no such features.

Data reduction not only reduces the amount of calculation but also serves the purpose of narrowing down the features. If the amount of data is too great, the number of features that are extracted also increases as a result, possibly so many that they cannot be realistically used. For example, if the number of the features extracted is more than 10000, there would be the question of how to use that many features. But if the amount of data is cut down without purpose, useful features may be lost. Thus, the amount of data is reduced efficiently with a minimal influence on the extraction of features when using a method whereby:

(1) Sections with a width n+1 are removed.

(2) Sections containing a discrete value of 0 and having a width of 2 or more are removed.

(3) Sections with a width of n′ or more are removed (n′<n).

The sections mentioned under condition (1) should be naturally eliminated, because they include all the sections and all of the values would create lines of value 1. Condition (2) deals particularly with sections (100 or less) represented by the discrete value 0. Experiments show that data is concentrated at values of 100 or less and other relatively low values. The sections eliminated by condition (2) would otherwise produce many 1s and therefore tend to be extracted as features. As mentioned in section 1.1. values of 100 or less can be regarded as showing no expression at all. It is pointless to consider sections combining sections having no expression at all and sections with values of 100 or more, that is those sections where expression can be thought to exist to some extent. It is a problem if too many relatively insignificant features are produced and, as a result, valuable features are rendered invisible. For this reason, the sections that fall under the above condition (2) are eliminated. Finally, condition (3) is given to deal with the fact that there are still too many features extracted even after data reduction through conditions (1) and (2). While condition (3) makes it impossible to extract features that are distributed evenly over a wide range of sections, features that exist in narrow sections in a concentrated manner are obviously more important, and it is valuable to consider such features. Although data can be reduced in size by selecting a smaller n′, this has to be chosen appropriately, as this may adversely affect the extraction of useful features as mentioned above.

The amount of data can be significantly reduced by conditions (1) to (3), while at the same time effectively narrowing down the features to be extracted.

3.2 Algorithm

A description of an algorithm for searching for all of the rules that can be the disease causation rule as defined in section 2.3 will follow.

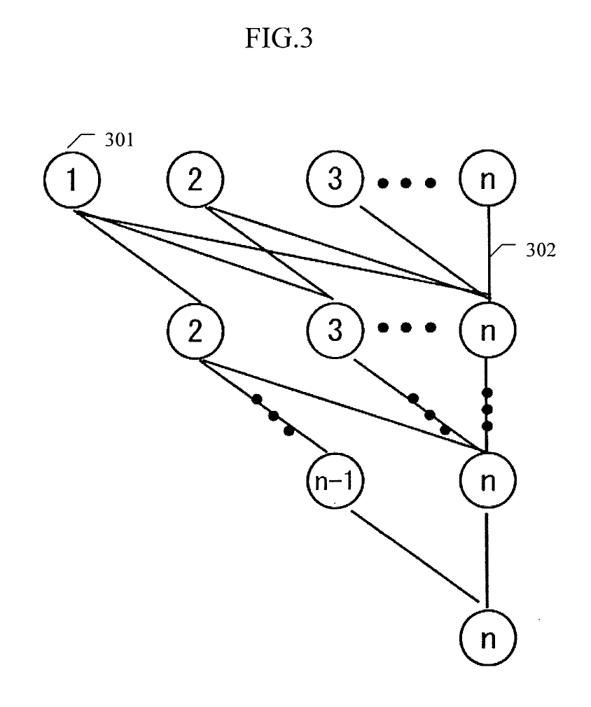

First, a combination of all the rules must be created. For this purpose, a search tree shown in FIG. 3 is considered. Starting from a root 301, a branch 302 extends downward. A node that is newly added to a path is different from any other nodes in the path. By thus considering paths of any desired lengths, any desired combinations of paths can be created. All of these paths can then be examined to see if there is a disease causation rule.

However, examining all imaginable rules to find a disease causation rule would mean having to examine a huge number of combinations. For example, supposing that the number of pairs of genes and their sections is 10,000 and when the length of a combined rule is limited to 5, the number of combinations still becomes ₁₀₀₀₀C₅, which is an unrealistic number. In order to avoid such an explosion of calculation amounts, an algorithm that is proposed here creates paths in an order of depth priority, so that those paths that do not satisfy the conditions for the disease-creating rule along the way are prevented from extending further.

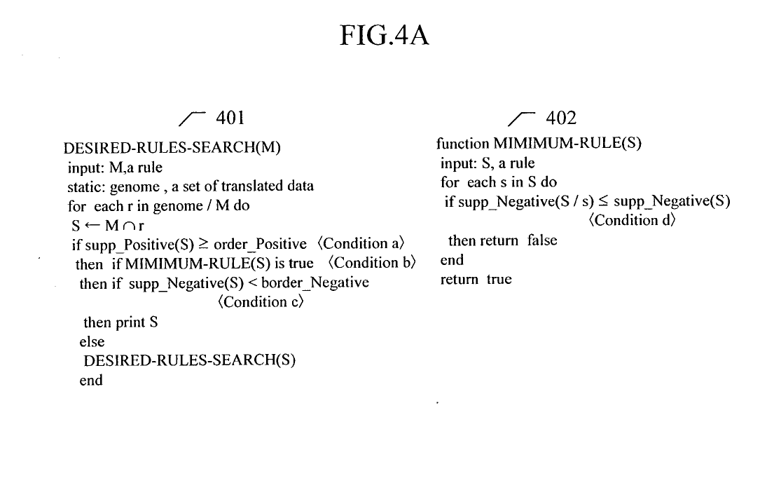

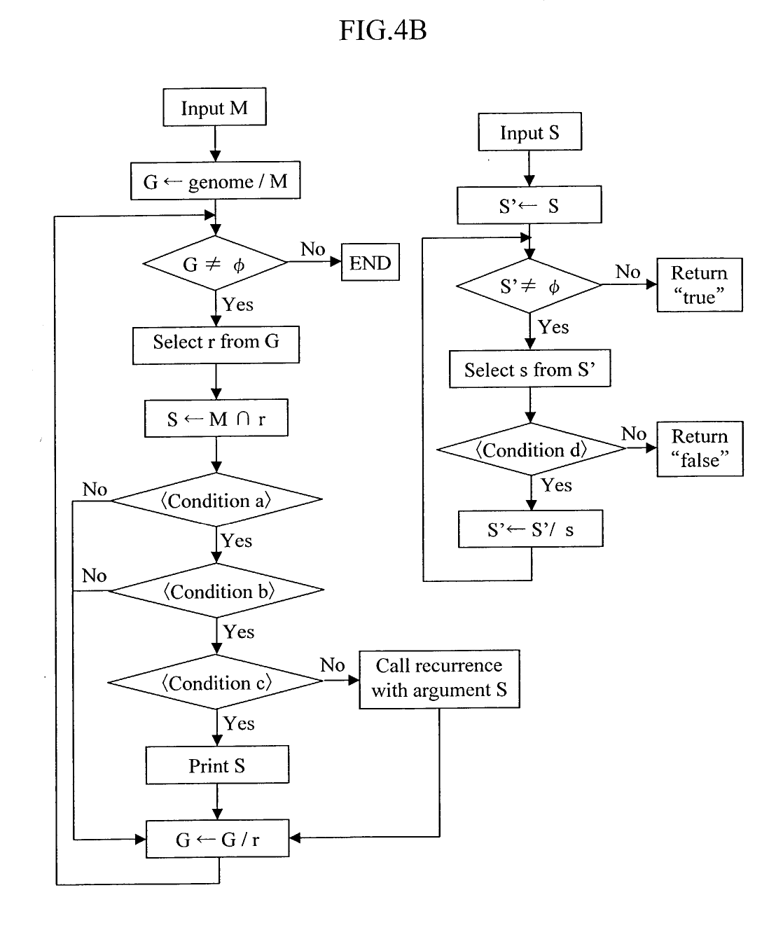

When this method is adopted, while wasteful calculations can be eliminated, it becomes necessary to judge whether a new combination constitutes a disease-creating rule each time such new combination is created. If too many calculations are allotted for this judgment, the overall amount of calculation would be excessive. Accordingly, the algorithm is designed such that the conditions imposed for a rule to become a disease causation rule are judged in an order of increasing amounts of calculation required. FIG. 4A shows an example of a program incorporating this feature. FIG. 4B shows a flowchart of the program.

In an algorithm 401 for determining a disease causation rule, a new rule S is created by adding, to an input rule M, a rule with the number of elements 1 from a set Genome of converted gene data which is not included in rule M, thus creating rules recursively. Before a recursion is iterated, the conditions for the disease causation rule are judged. The judgment of the minimum gene rule is conducted at the beginning under the conditions related to #spt_{Positive}(R), because this judgment requires more calculation than the other judgments do. The amount of calculation for the condition relating to #spt_{Negative}(R) is also small. But even if this judgment is brought to the beginning, the judgment of the minimum gene rule is necessary after all. Thus, the judgment of the minimum gene rule is conducted at the beginning for simplifying the description of the algorithm.

The judgment of the minimum gene rule is conducted in an algorithm 402, in which the amount of calculation is reduced according to the above-described theorem.

4. Diagnostic Support System

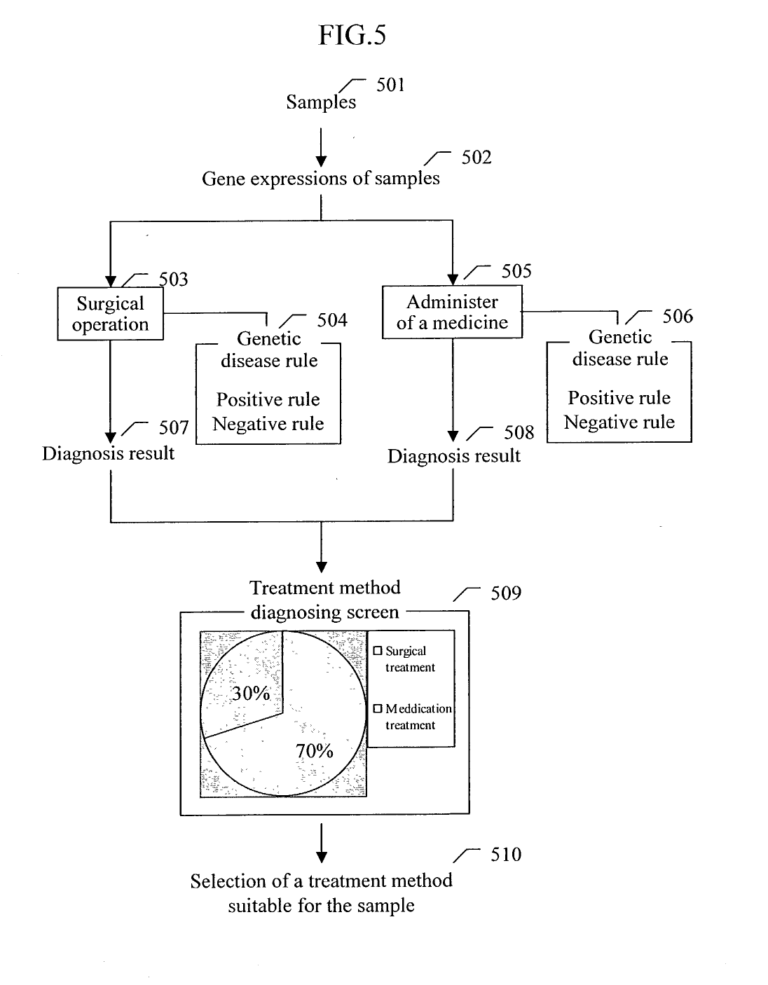

A method of numerically expressing the probability of a new sample belonging to either group by using the disease causation rule extracted in section 3 will be described (FIG. 5).

By applying the algorithm described in section 3 to data given in which positive and negative samples are exchanged, a disease causation rule for Positive Group (Positive rule) and a disease causation rule for Negative Group (Negative rule) can be obtained. By using these rules as a database 504, a diagnostic system 503 is constructed. When a database 506 and a diagnostic system 505 are similarly constructed based on other data, a more effective diagnostic system can be constructed by using these multiple diagnostic systems simultaneously. In each diagnostic system, diagnosis is made in the following manner.

The sums of the differential confidence values of the disease causation rules in the database are expressed by C_{Positive} and C_{Negative} for Positive and Negative Groups. Gene expression levels of a new sample 501 is measured (502). The sums of the differential confidence values of those rules of the extracted disease causation rules that exist in the new sample are expressed by C′_{Positive} and C′_{Negative}. The rules that exist in the sample refer to those rules of the disease causation rules in which the expression levels of the genes in the sample satisfy the conditions. By using these rules, the ratios of the disease causation rules that are satisfied by the new sample are defined as follows:

$${R}_{Positive} = \frac{{{C}^{′}}_{Positive}}{{C}_{Positive}} , {R}_{Negative} = \frac{{{C}^{′}}_{Negative}}{{C}_{Negative}}$$

Based on these definitions, the relative probabilities P_{Positive} and P_{Negative} of the new sample belonging to Positive or Negative Group are expressed as follows:

$${P}_{Positive} = \frac{{R}_{Positive}}{{R}_{Positive} + {R}_{Negative}}$$

$${P}_{Negative} = \frac{{R}_{Negative}}{{R}_{Positive} + {R}_{Negative}}$$

By comparing P_{Positive} and P_{Negative}, an investigation into which group a new sample is more likely to belong to can be performed. For example, if data given is divided into a group of samples that showed improvements after a certain medicine was administered, and a group of samples that did not show any such improvements, whether or not the medicine should be administered in the future can be determined by the diagnostic system of the invention (507). Alternatively when other data is given about the effects of performing a particular surgery, the diagnostic system can similarly provide results (508) as to whether or not such a surgery should be performed. By normalizing a plurality of P_{Positive} values obtained from these results of diagnosis such that their sum is 1, how recommendable a particular treatment method is can be shown (509). The level to which a treatment can be recommended can thus be expressed numerically or by using graphs. In the illustrated example, the degree of recommendation for surgical treatment is 70%, while that for medication is 30%. Based on these results, an effective treatment method, such as by medication only or a combination of medication and surgery, can be selected (510).

While the example of FIG. 5 comprises a surgical treatment diagnostic system 503 and a medication treatment diagnostic system 505 individually, a single diagnostic system may be employed to provide surgical treatment diagnosis and medication treatment diagnosis based on databases 504 and 506, respectively. More than two databases may also be employed in which separate genetic disease rules are stored, so that the selection of a treatment method can be facilitated based on more than two references.

Other examples of properties that can be diagnosed by the present diagnostic system include the property that a certain treatment method will be effective, that the sample is afflicted with a certain disease, and that the sample is liable to develop a certain disease.

5. Selection of the Subject by Ontology

While the algorithm described in section 3 can reduce the amount of calculation for feature extraction greatly, there still remains a great amount of calculation to be done because the original amount of calculation, that is the total number of combinations, is so great. To eliminate this problem at its source, the original calculation amount has to be reduced, and for this purpose it is effective to narrow down the genes used as subjects to some extent. The total number of combinations can be expressed as 2^{k} where k is the number of the genes. Namely, it will be seen that, theoretically, the amount of calculation can be reduced by half by reducing the number of the genes just by one. It will also be seen that by further reducing the number of genes by h, the amount of calculation is narrowed down to ½^{h}, This means that, for example, the amount of calculation can be reduced to 1/1024, 1/1048576 and 1/1073741824 by reducing the number of genes by 10, 20 and 30, respectively. Thus, by narrowing down the genes that are used for feature extraction a great effect can be obtained at the expense of a very small amount of loss. Although the loss is very small, the elimination of some genes under investigation could result in failure to extract important features that should have been extracted. Accordingly, the genes are narrowed down by classifying the genes by ontology.

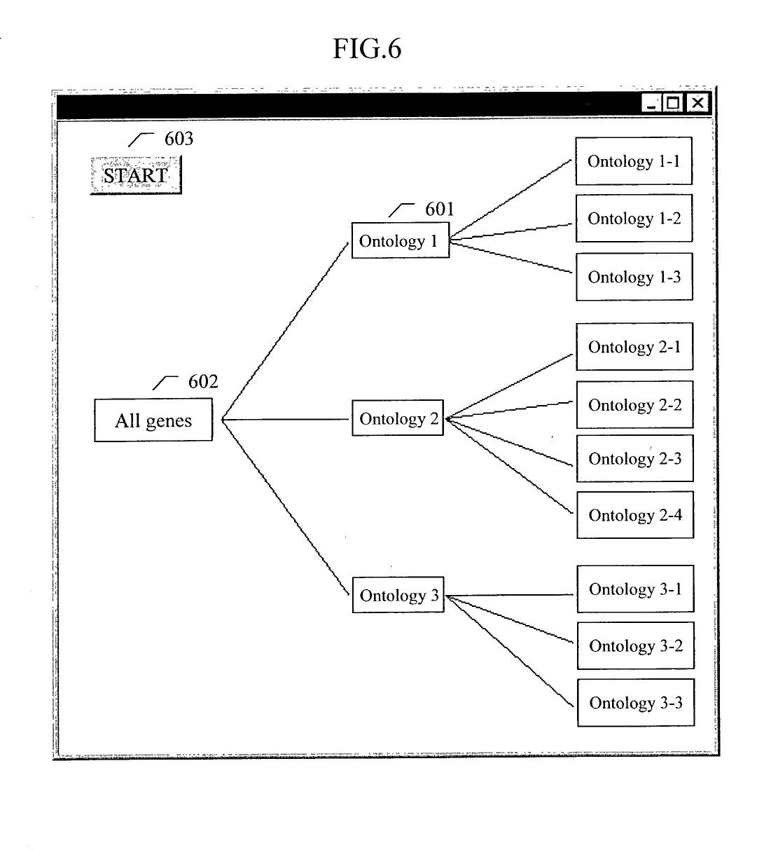

The classification of genes by ontology is based on a variety of factors, and the classification has a hierarchical structure (FIG. 6). The user selects valid genes from this classification based on various kinds of information and uses them as objects to be processed by the algorithm. By so doing, the above-mentioned risks can be minimized.

Using software, a tree structure shown in FIG. 6 is drawn based on the classification by ontology. The user selects an item 601 by clicking from the drawing that he or she thinks is related to a particular disease. When related items are not narrowed down, all of the genes can be selected by clicking on an “All genes” button 602. Thereafter, by pushing a start button 603 in the upper left portion of the screen, the algorithm is activated based on the selected classification. If the start button is pushed without making the selection, the algorithm is activated based on all of the genes.

6. Numerical Expression of the Importance of Genes

All of the disease causation rules defined in section 2 are derived by the algorithm described in section 3. The disease causation rules, as combinations of genes, strongly characterize a group with a certain property. While this is very significant data for determining to which group a new sample is more likely to belong, it does not provide much information about the individual genes. In actual situations, it is important not only that a new sample can be accurately judged but that it can be clearly shown what genes are responsible for the particular property of the sample. Accordingly, a method is considered by which the importance of each gene can be derived from a disease causation rule that is extracted and the degree of contribution of each gene to the particular property can be examined.

A gene that appears in many rules is more important than another gene that appears in hardly any rules. Also, a gene that appears in rules with higher differential confidence is more important. Thus, the importance of a gene is expressed as the sum of the values of differential confidence of the rules in which the gene appears.

$$Importance of gene g = {\sum }_{Rule R including g}^{} Differential confidence of R$$

As it can be considered that there is more than one gene related to any particular disease, it is necessary to consider the mutual connections between genes, as well as determining the importance of each gene. From this viewpoint, it is supposed that the connection between two genes that appear in a rule at the same time is strong. Thus, the sum of the differential confidence values of rules in which certain genes g1 and g2 appear is considered as the degree of connection of g1 and g2.

$$Degree of connection of genes g1 and g2 = {\sum }_{Rule R including g1 and g2}^{} Differential confidence of R$$

7. Visualization

Java-based viewers are used for letting the user know the extracted rules, important genes and correlations among genes clearly. There are four viewer windows as indicated below. The viewer windows can be dynamically modified by varying the parameters of the algorithm on each panel. Thus, the user can see subtle changes in the importance of a gene or the correlation among genes as the parameters are varied.

7.1 Visualization of the Rules

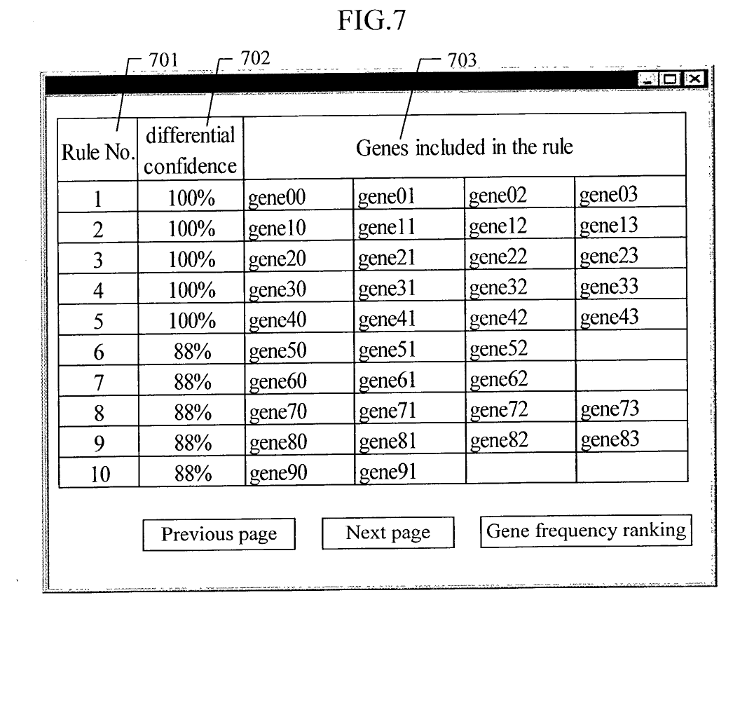

A feature dividing the two groups of Positive and Negative Groups is visualized by means of the distribution of expression levels, which provides evidence of the differences between the groups. Generally, more than one candidate for the feature dividing the two groups can be obtained. FIG. 7 shows a viewer window for displaying a list of extracted rules. Each line of the list corresponds to an individual rule extracted. A column 701 indicates the identification number of the extracted rule. A column 702 indicates the differential confidence of each rule. In the illustrated example, the list is arranged in a decreasing order of differential confidence. A column 703 shows genes that are included in the individual rules.

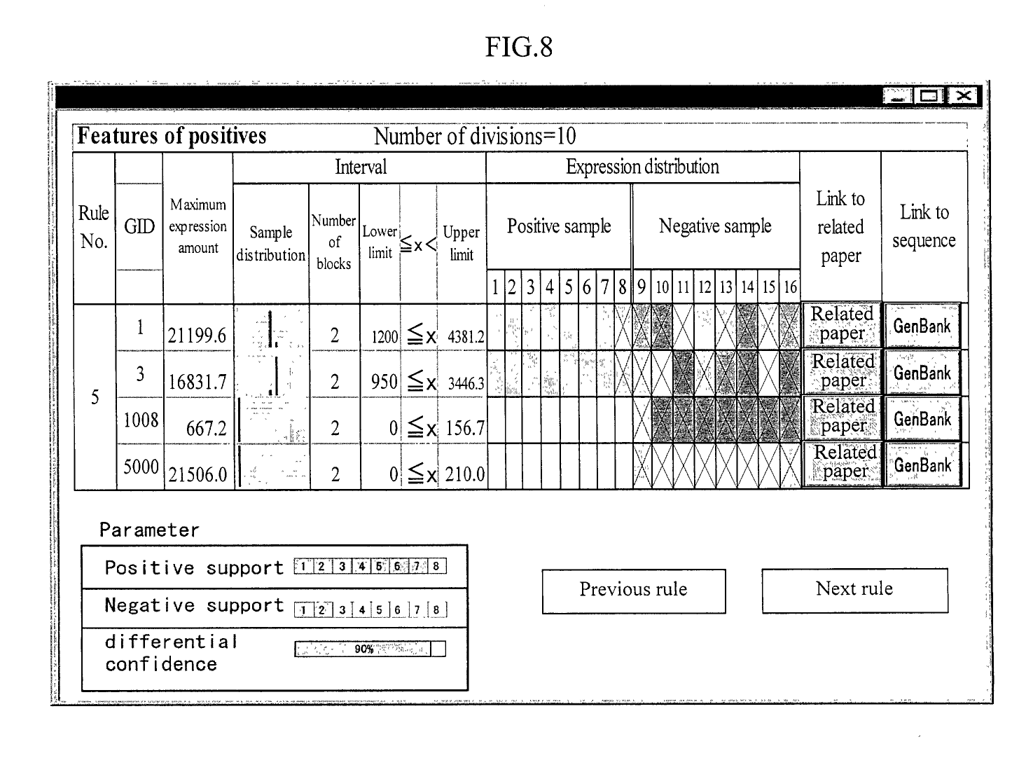

FIG. 8 shows an example of the viewer window for displaying the features of a rule. This window can be opened by selecting a line of the rule in the list viewer window of FIG. 7. The viewer of FIG. 8 visualizes rule No. 5 of the list of FIG. 7. “Number of divisions” indicates the number of divisions (sections) between a threshold value and the maximum expression level value. In the illustrated example, “Number of divisions=10” indicates that the interval between the maximum value and 100 of expression level has been equally divided in 10 during the conversion of the expression level data into discrete values by means of the Border function, as described in section 1.2. As shown in the parameter column 807, this rule has a Positive support of 7 or more, a Negative support of less than 2, and differential confidence of 90% or more.

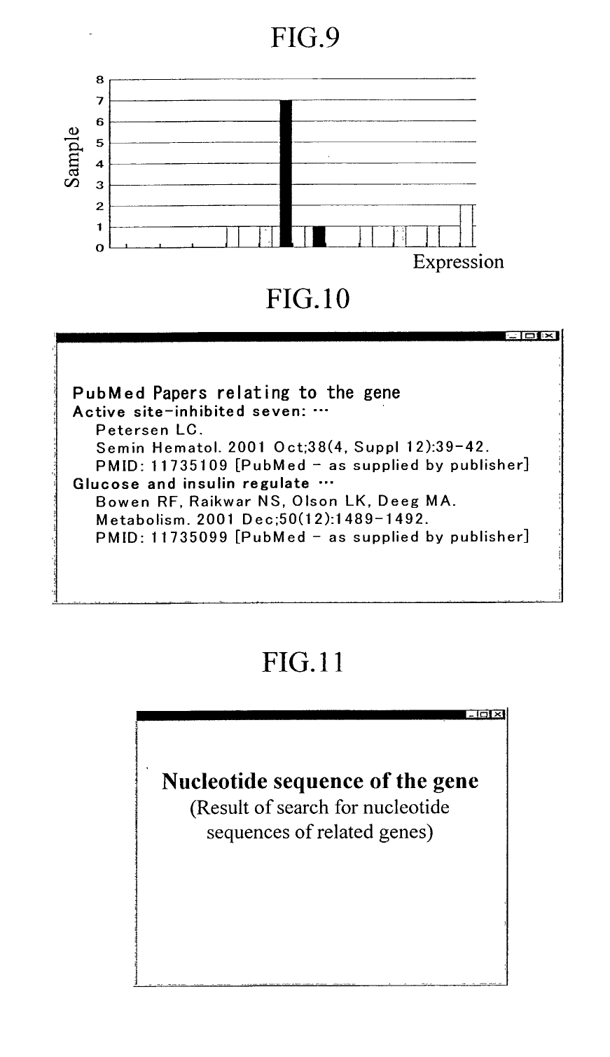

In the drawing, individual lines indicate genes constituting the rule No. 5 and their expression level sections. GID is a unique identification number for each gene. The column “Maximum expression level value” shows the maximum value of the expression level of this gene taken from among the samples in the database. In the column “Lower limit≦×<Upper limit,” the lower and upper limits of the interval are indicated by specific values. The “Number of blocks” column indicates the number of sections of expression levels under consideration. The “Distribution of samples” graph shows a bar graph indicating the expression level on the horizontal axis and the number of samples on the vertical axis. How many subjects there are in which block is indicated by bars with a darker shade for Positive and by bars with a lighter shade for Negative. An enlarged view is shown in FIG. 9. As shown, the location of the range of this rule in the interval between 0 and the maximum value of the expression levels can be shown in a clear and easily recognizable manner.

The bars in the “Distribution of expression levels” graph are given different shades to indicate the level of expression levels of sample genes. The lighter shades indicate expression levels that are closer to zero, while darker shades indicate expression levels closer to the maximum value. The samples marked by X indicate that the genes fail to satisfy the rule. With regard to the samples belonging to Positive Group, by showing that those samples in the sections of the illustrated gene expression levels are not necessarily present in the sections with regard to the samples belonging to Negative Group, the user can easily see that the two groups are divided on the basis of this rule.

The “Related papers” button and “GenBank” button on the right are for links to PubMed, an official database of papers on the individual genes, and to GenBank, a database of nucleotide sequences. By clicking on the button “Related papers,” information about related publications is indicated in a window as shown in FIG. 10. By clicking on the button “GenBank,” the nucleotide sequence of a gene (not shown) is indicated in a window as shown in FIG. 11. Thus, the user can discover the details about a particular gene. By clicking on a “Next rule” button, a next-level rule can be seen, and by clicking on a “Previous rule” button, an upper-level rule can be seen.

7.2 Visualization of the Important Genes

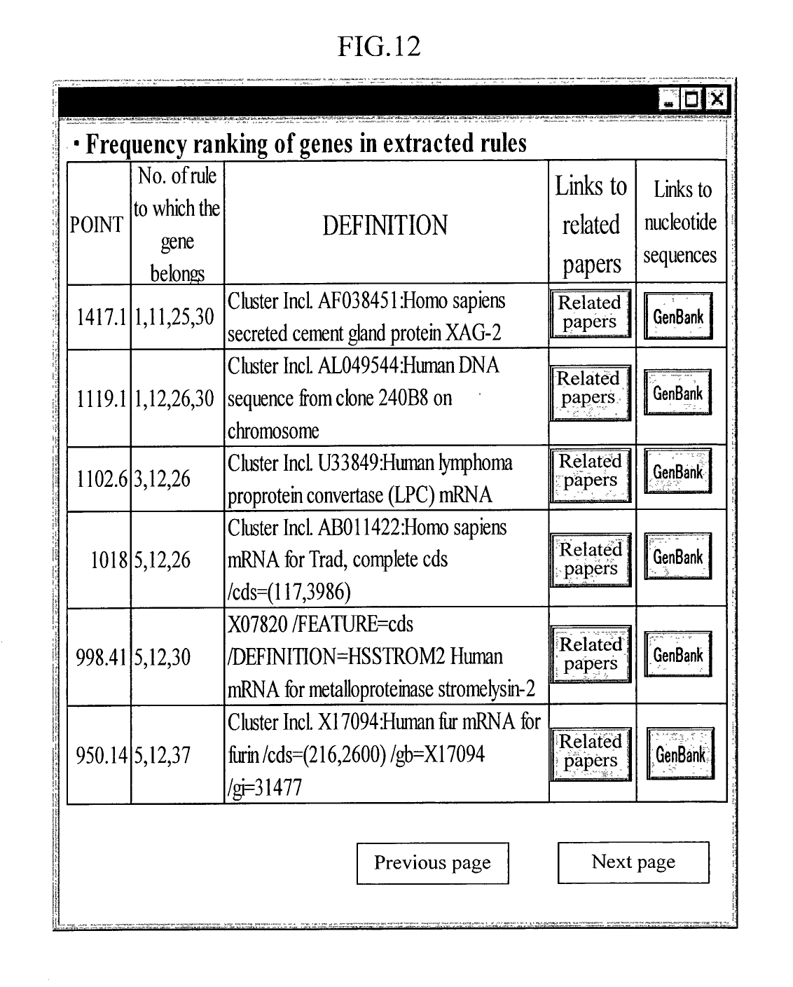

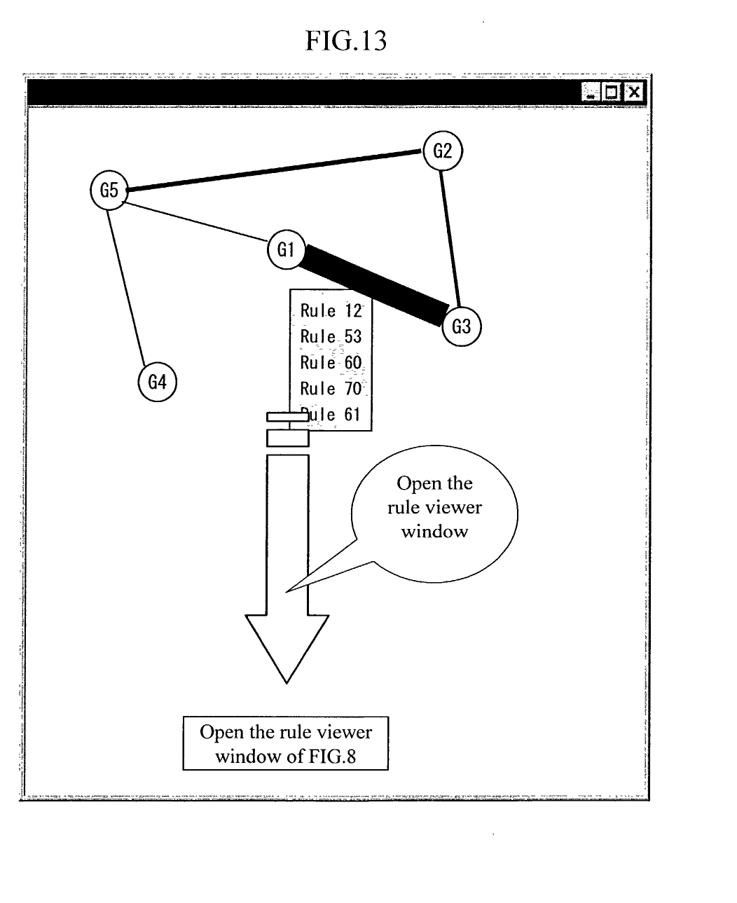

By calculating the importance of the genes that appear in a rule, the genes are rearranged in an order of importance and displayed. FIG. 12 shows an example. In the viewer of FIG. 7, by clicking on a “Gene frequency ranking” button, the viewer of important genes of FIG. 12 is shown.

In the viewer of FIG. 12, each line represents a gene, with the genes arranged in an order of increasing importance towards the top. In the drawing, “POINT” indicates the importance of the gene. “Rule No. to which the gene belongs” indicates the number of the rule (see FIG. 8) to which the gene of that line belongs. By clicking on the number indicated in the Rule No. column, a corresponding rule as shown in FIG. 8 can be displayed. Further, there is a “DEFINITION” section where the name of the gene is indicated. By clicking on the “Related paper” button, information about official papers related to that gene can be seen in a window, as shown in FIG. 10. By clicking on the “GenBank” button, the nucleotide sequence of the gene (not shown) is displayed in a window as shown in FIG. 11, enabling the user to learn the details about the gene instantly. Furthermore, by clicking on the “Next page” button, a lower-level gene can be seen, and by clicking on the “Previous page” button, an upper-level gene can be seen.

7.3 Visualization of the Degree of Connection of Genes

As shown in FIG. 13, the degree of connection of genes that appear in a rule is calculated, and a network is constructed of the calculated degrees and shown in a graph. Thus, the user can easily understand which gene is connected with which gene. Each node of the graph represents a gene, with each line representing the degree of connection between the genes at both ends of the line. The greater the degree of connection between any two genes, the more emphasis the associated line is given. In the illustrated example, it will be clearly seen that genes G1 and G3 are strongly connected, while genes G1 and G4 are not related at all. By clicking on a line, a viewer 1303 of a rule can be called up in which the two genes corresponding to the nodes at both ends of the line appear simultaneously. In the drawing, the viewer shows a rule in which G1 and G3 appear simultaneously. In addition, the positions of the nodes shown are calculated such that the user can easily recognize the relationship between the genes. Thus, the individual lines can be shown in such a manner that they do not overlap with one another and the emphasized line is located at the center.

7.4 Coordination With a Network Formed by Correlations of Genes Appearing in Literature

A graph of a network formed by binary relations of genes appearing in papers about genes is drawn simultaneously with the network formed by combinations of important genes. By looking at two different networks simultaneously and thus visually recognizing the connection of genes commonly appearing in the networks. the user can expand his or her understanding about the genes characterizing a group.

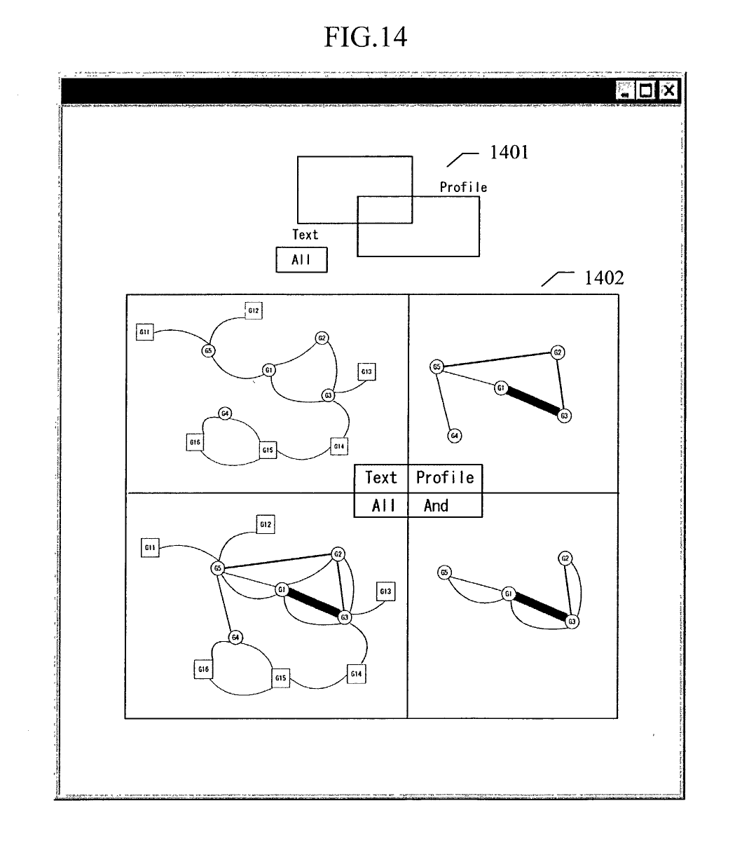

FIG. 14 shows an example of a display of the network indicating the connection of genes shown in FIG. 13 in association with the network formed by correlations of genes appearing in papers. The square nodes that are newly added represent the genes in the papers, with the curved lines indicating the relationships among genes in a network formed by binary relationships of genes appearing in the papers about genes.

A graph 1402 can be varied with a panel 1401 at the top in which two regions named “Text” and “Profile” are drawn. By clicking on the Profile region, the Text region, or their common portion the graph 1402 corresponding to the following networks is dynamically drawn.

(1) A network formed by the degrees of connection of genes (Profile).

(2) A network formed by the correlations among genes appearing in papers (Text).

(3) A network of two combined networks (All).

(4) A network formed by the superposed portion of the two networks (And).

8. System Configuration

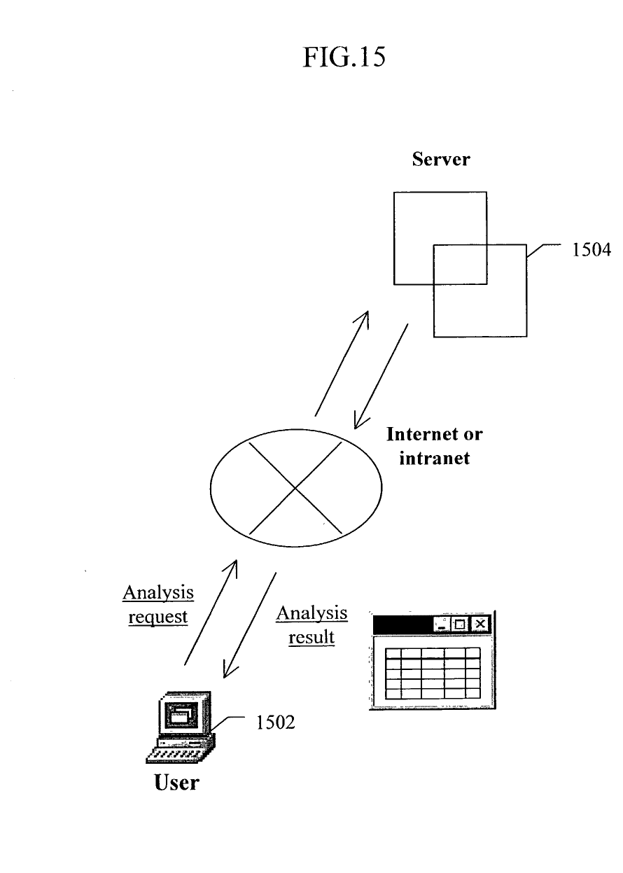

A series of processes from the request for data analysis to the visualization of results is performed over the Internet or an intranet (FIG. 15). The Internet is selected for data that may be publicly disclosed, while an intranet is selected for highly confidential data. A user 1502 sends an analysis request to the server via an appropriate network. In response, the server 1504 performs the requested analysis and displays the results for the user. Thus, the user can easily analyze the latest data on a large scale.

Thus, in accordance with the invention, the genetic difference between a group with a certain feature and another group without the feature can be extracted and visualized. Accordingly, estimations can be made as to which group a new sample belongs to, thereby making it possible to provide effective treatment.

What is claimed is:

1. A display system for displaying information about expression levels of genes in samples, wherein information about expression levels of each of a plurality of samples belonging to a first group is displayed comparatively with information about the expression levels of each of a plurality of samples belonging to a second group that has a different property from that of the first group.

2. The display system according to claim 1, wherein items of information about the expression levels of a plurality of genes are displayed in a comparative manner.

3. The display system according to claim 1, wherein the information about the expression levels indicates whether or not the expression levels are within a predetermined range.

4. The display system according to claim 3, wherein the first group has a specific property, while the second group does not have that specific property.

5. The display system according to claim 1, wherein items of information about the expression levels of the multiple samples belonging to the first group are displayed adjacent to one another, and items of information about the expression levels of the multiple samples belonging to the second group are displayed adjacent to one another.

6. A display system for displaying information about the expression levels of genes in samples, wherein two histograms are displayed, wherein the first histogram is prepared based on the expression levels of each of the samples belonging to a first group, and the second histogram is prepared based on the expression levels of each of the samples belonging to a second group with a different property from that of the first group, each histogram having one axis showing the expression levels and the other axis showing the number of samples.

7. The display system according to claim 6, wherein the first and second histograms are displayed in a superposed manner on a single graph sharing the same axes.

8. The display system according to claim 7, wherein the first and second histograms are displayed in different ways.

9. The display system according to claim 6, wherein the axis indicating the expression levels is divided into a plurality of expression level sections.

10. The display system according to claim 6, wherein the first group has a specific property while the second group does not have the specific property.

11. A medical diagnostic support system comprising:
a storage unit for storing a set of combinations of expression level ranges of a plurality of genes characterizing the presence of a specific property, and a set of combinations of expression level ranges of a plurality of genes characterizing the absence of the specific property;
a computation unit for computing the probability of a tested sample having the specific property by comparing the expression level ranges of a plurality of genes of the tested sample with combinations of expression level ranges of the plurality of genes stored in the storage unit; and
a display unit for displaying the results of computation in the computation unit.

12. The medical diagnosis support system according to claim 11, wherein the specific property relates to the fact that a specific treatment method is effective.

13. The medical diagnosis support system according to claim 11, wherein the specific property relates to the fact that the sample is afflicted with a specific disease.

14. The medical diagnosis support system according to claim 11, wherein the specific property relates to the fact that the sample is prone to a specific disease.

15. The medical diagnosis support system according to claim 11, wherein the result of computation in the computation unit is displayed on the display unit in numerical terms.

16. The medical diagnosis support system according to claim 11, wherein the result of computation in the computation unit is displayed on the display unit in percentage terms on a graph.

17. The medical diagnosis support system according to claim 11, wherein the storage unit for storing a set of combinations of expression level ranges of a plurality of genes characterizing the presence of a specific property, and a set of combinations of expression level ranges of a plurality of genes characterizing the absence of the specific property is provided for each of a plurality of different properties.

18. The medical diagnostic support system according to claim 17, wherein the computing unit calculates the probability of the tested sample having a first property by comparing the expression level ranges of a plurality of genes in the tested sample with combinations of expression level ranges of a plurality of genes stored in a first storage unit, and calculates the probability of the tested sample having a second property by comparing the expression level ranges of a plurality of genes in the tested sample with combinations of expression level ranges of a plurality of genes stored in a second storage unit.

19. The medical diagnostic support system according to claim 18, wherein the display unit displays the probability of the tested sample having the first property and the probability of it having the second property.

20. The medical diagnostic support system according to claim 18, wherein the display unit displays the probability of the tested sample having the first property and the probability of it having the second property in percentage terms on a graph.
